(19) United States
(12) Patent Application Publication
(10) Pub. No.: US 2006/0292690 A1
(43) Pub. Date: Dec. 28, 2006

(54) Method of making cell growth surface

(75) Inventors: Tzu-Ying Liu, Jhubei City (TW); Ing-Kae Wang, Hsinchu (TW); Sing-Ying Hsieh, Hsinchu (TW); Lewis Ho, Waterford, CT (US); Yu-Chi Wang, Jhubei City (TW); King-Ming Chang, Hsin-Chu (TW)

Correspondence Address: BIRCH STEWART KOLASCH & BIRCH, PO BOX 747, FALLS CHURCH, VA, 22040-0747, US

(73) Assignee: CESCO Bioengineering Co., Ltd.

(21) Appl. No.: 11/471,576

(22) Filed: Jun. 21, 2006

Related U.S. Application Data
(60) Provisional application No. 60/694,183, filed on Jun. 22, 2005.

(51) Int. Cl. C12N 5/06

(52) U.S. Cl. 435/325; 435/404

(57) ABSTRACT

The present invention discloses a three-dimensional porous growth surface made from polysaccharide material, especially the alginic acid, to enhance cell growth surface, promote cell adherence, immobilization and propagation, maintain surface structure integrity, enable programmable degradation, and thus increase cellular production. The present invention teaches several methods: a method to enhance the integrity of the growth surface by protecting the growth surface in a rigid solid support; a method of use for enhancing the performance of the surface; and a method of modifying a growth surface for eukaryotic and/or prokaryotic cells comprising the steps of increasing surface area by creating porous and 3-D structure, treating a surface to encourage cell attachment, promoting cell growth and proliferation, and disposing the growth surface in any conventional cell cultivating device. The growth surface is able to program degradation and release the cell/tissue mass after the culture is completed.

### CROSS REFERENCE TO RELATED APPLICATIONS

The present application claims benefit of U.S. Provisional Application No. 60/694,183 filed Jun. 22, 2005.

### BACKGROUND OF THE INVENTION

1. Field of the Invention

The present invention relates to a growth surface and structure for culturing cells and the method of making the same, and more particularly, to a growth surface and structure for culturing cells followed with cells harvest and the method of making the same.

2. Description of the Prior Art

Revolutionary advances in biotechnology and genetic engineering have created high demand to market cellular products, such as protein pharmaceuticals, cytokines, interferon, monoclonal antibodies, hormones, growth factors, insulin, viral products, vaccines, nucleic acids, enzymes, and cells and/or tissues for transplantation. The demand of these products has thus created an ever-increasing need for efficient and economic methods of production.

Eukaryotic cells such as mammalian cells have become most popular for providing high quality and quantity of efficacious protein cellular products. Culturing mammalian cells has long been used to produce vaccines, genetically engineered proteins, pharmaceuticals and other cellular products. Generally, eukaryotic cells can be anchorage-dependent, anchorage-independent or both. However, eukaryotic cells are generally anchorage-dependent, thus requiring a growth surface to anchor, mature and produce desired cellular products. Examples of anchorage-dependent cells are fibroblasts, epithelial cells and endothelial cells. Eukaryotic cells such as lymphocytes, some transformed cells and some cancer cells are “anchorage-independent” cells and can grow in suspension. Regardless of their type, most eukaryotic cells in culture have the following characteristics in common and these characteristics play a key role in designing an efficient growth surface and cultivating device.

The attachment of anchorage-dependent cells to a growth surface is the key to cell vitality and fundamental to all types of culture techniques including but not limited to traditional mono-layer culturing or culturing with a carrier and/or micro-carrier system. Since the proliferation of anchorage-dependent cells can only occur after adhesion to a suitable growth surface, it is important to use surfaces and culture procedures which promote cell adhesion. Cell adhesion includes adsorption of attachment factors such as proteins to a cultivation surface, contacting the cells with the cultivating growth surface, attaching the cells to a treated surface suitable for cell adhesion, spreading and replicating the adhered or attached cells across the growth surface until these cells come into contact with another surface-growing cell (i.e., “contact inhibition”).

In order to have a viable anchorage-dependent cell culture, the culture needs an appropriate cultivating growth surface or carrier, a mechanism for circulating culture medium particular to the cell type to be cultured and proper aeration with an adequate supply of gas to support and maintain cell growth. There are several different ways to culture cells and they are batch system in which nutrients are not replenished during cultivation although oxygen is added as required, fed batch systems in which nutrient and oxygen are monitored and replenished as necessary and perfusion systems in which nutrient and waste products are monitored and controlled with continuous replenishment of fresh medium.

There are several types of cultivation carriers that are currently known in the art. For example, dextran-based (e.g., Cytodex I, DEAE-dextran and Cytodex III, porcine collagen-coated dextran; Amersham-Pharmacia, UK) or coated polystyrene-based (e.g., SoloHill, U.S.) microcarrier. Microcarriers are typically very small and have diameters of approximately 50 to 250 micrometers, although larger or smaller sizes of microcarriers have been used (U.S. Pat. No. 5,114,855 issued May 19, 1992 to Hu et al.). A second type of cell-cultivation carrier includes a porous matrix material made from ceramics, polyurethane foam, or polyethylene terephthalate (PET), or biodegradable material from poly(lactic-co-glycolic acid) (PLGA), collagen, chitosan. Example products are PET based (BioNOC II carriers from CESCO Bioengineering, Taiwan, and FibraCel disks from New Brunswick Scientifics, U.S.)

Cell cultivation carriers can also be categorized according to its surface property. For example, there are non-porous or poreless and porous carriers. The porous carriers are generally more advantageous than the non-porous carriers since the porous carrier provides a bigger surface-to-volume ratio as well as the protection to insulated cells. Because of its porous nature, these carriers form multiple three-dimensional cavities within the growth surfaces and thus maximizes cell attachment and also protect cells from being dislodged and/or damaged from shearing stress resulted from aeration, agitation and impact during the feeding and/or harvesting processes.

Many cell-cultivating systems currently available in the art employ microcarriers that are porous and/or nonporous or poreless. These microcarriers such as microcarrier beads currently available are used in anchorage-dependent cell production systems. These microcarriers must be used in conjunction with a stirring equipment and/or aeration capability. However, a common problem with microcarrier systems is that the stirring action required to sustain the cell culture can damage or even kill the cells thereby decreasing the efficiency of the cultivation system and the production of the desired cellular product.

Microcarrier systems can also be fabricated in small spheres from an ion exchange gel, dextran, polystyrene, polyacrylamide, or collagen-based material. These materials have been selected for their compatibility with cells, resilience to agitation and specific gravities that can maintain the microcarriers suspended in the growth media. Microcarriers are generally kept in a growth medium suspended with gentle stirring within a vessel in order to ensure equal distribution of nutrients and air to all cells. Microcarrier system is currently considered to be the most suitable system for large-scale cell culture because it has the highest surface-to-volume ratio and enables even distribution of nutrients to cells.

Nevertheless, current microcarrier culture system has serious disadvantages. These disadvantages include high cost and high cell mortality rate due to exposing to high level of shearing forces caused by stirring and aeration during cultivation. Most commonly used microcarriers utilize porous non-rigid dextran as a support matrix. This compressible matrix is thought to reduce potential damages to the microcarriers and their attached cells when the microcarriers collide in agitated reactors (Microcarrier Cell Culture: Principles and Methods, Pharmacia Fine Chemicals, Uppsala, Sweden, pages 5-33 (1981)). These porous microcarriers, however, also have serious disadvantage in retaining cellular products that results in the adsorption of growth factors and other components from the medium (Butler, M., “Growth Limitations in Microcarder Cultures”, Adv. Biochem. Eng./Biotech. 4:57-84 (1987)).

U.S. Pat. No. 5,015,576 issued May 14, 1991 to Nilsson et al. relates to making particles which enclose cavities by adding a water-insoluble solid, liquid or gaseous cavity generating compound to an aqueous solution of matrix material. Subsequent to forming particles by dispersion in a water-insoluble dispersion medium, the matrix is rendered insoluble in water by cooling, covalent cross-linking or by polymerization. The cavity-generated compound is washed out, thereafter the particles can be used as ion exchangers in gel filtration processes, in hydrophobic chromatography or in affinity chromatography, optionally subsequent to derivatizing the particles. The particles can also be used as microcarriers for cultivating anchorage-dependent cells.

U.S. Pat. No. 5,385,836 issued Jan. 31, 1995 to Kimura et al. relates to a carrier for animal cells attachment during cell culturing or for immobilization of animal cells. This carrier is produced by coating a porous substrate with a cell adhesive material in the form of a mixture containing chitosan. The porous substrate is a non-woven fabric prepared by impregnating a non-woven fabric web with a binder resin which contains silk fibroin, gelatin and chitosan. Coating is carried out by contacting the non-woven fabric with a solution prepared by adding silk fibroin and gelatin to an acidic aqueous solution of chitosan to coat the non-woven fabric, drying the coated non-woven fabric and treating the dried non-woven fabric with an alkali to render the chitosan insoluble.

U.S. Pat. No. 5,565,361 issued Oct. 15, 1996 to Mutsakis et al. relates to a bioreactor having a motionless mixing element with attached cells method for the enhanced cultivation and propagation of cells in a bioreactor. The bioreactor has a housing and a motionless mixing element, the attachment of cells to the mixing element and a nutrient composition permitting attached cells to grow and divide. The motionless mixing element and the bioreactor have a porous, fibrous sheet material such as a corrugated or knitted woven wire material, such as stainless steel or titanium, and predetermined dimensions for the height and diameter of the fiber in order to provide a maximum surface area for the attachment of the cells to be cultivated.

U.S. Pat. No. 5,739,021 issued Apr. 14, 1998 to Katinger et al. relates to a porous carrier for biocatalysts with a water-insoluble inorganic filler and a polyolefine binder selected from polyethylene and polypropylene, has open pores to allow cells to penetrate and grow within its pores. The density is above 1 g/cm³.

U.S. Pat. No. 6,214,618 issued Apr. 10, 2001 to Hillegas et al. relates to a method of making microcarrier beads by forming a bead made of a lightly crosslinked styrene copolymer core with functional groups on the surface of the bead and washing the microcarrier beads with basic and acidic solutions to make the beads compatible for cell culture. The microcarrier bead can also be made of a styrene copolymer core with a tri-methylamine exterior which has been washed in basic and acidic solutions to make the beads compatible for cell culture.

Notwithstanding the variety of carriers taught in the foregoing art for cell cultivation, none of the carriers is capable of programming degradation and allowed releasing cells easily while retaining high surface-to-volume ratio and cell-adhesion properties for cell cultivation.

With rapid progress of biotechnology, any cell culturing technology either for prokaryotic cells or eukaryotic cells has been becoming increasingly important. Generally, eukaryotic cells are slow growing and vulnerable to injuries caused by shear stress and contamination. Majority of the eukaryotic cells are anchorage-dependent and require a growth surface for them to adhere and grow. In order to accommodate of this kind of eukaryotic cell cultures, various carriers with growth surfaces have been developed. Currently most available carriers are smooth surface carriers made on dextran-based material, porous matrix made by polyurethane or polyethylene terephthalate, and semi-permeable membrane such as hollow fibers made by polysulfone or cellulose acetate. However, the harvest of cells from those carriers is tedious, susceptible to contamination, and often is nearly impossible, especially for the carriers with porous structure. Therefore, the scale up for anchorage-dependent cells has been a slow, labor intensive, and expensive process. Because of this, there is strong need to develop a culture carrier which may solve this cell harvesting problem.

There are two major types of carriers for anchorage-dependent cells including particulate smooth surface carriers (nonporous or poreless) and porous carriers. The smooth surface does not lend itself to a large growth surface area and thus limits the number of cells to be adhered and grown. The porous carrier on the other hand provides at least one three-dimensional cavity to house cells. The porosity of the carriers also creates additional surface areas for cell anchorage that protect cells from being in direct contact with shear stress created by aeration, agitation and feeding. However, the task of harvesting cells from the porous carriers is often very difficult.

Carriers made by alginic acid (or alginate) have long been practiced for cell immobilization. However, the preparation of the immobilization process under sterile condition is difficult and usually limited for anchorage-independent cells. Alginate carriers are easy to be dissolved by adding chelating agent such as Ethylene-diamine-tetraacetic acid (EDTA), or sodium citrate. After the carriers are dissolved, cells can be released easily. Therefore, it could be a potential material for cell or tissue harvest.

Using alginate carriers for cell/tissue culture, there are three major problems: 1. Due to limitation of mass transfer inside of conventional alginate bead for immobilizing cells, cell density and viability can be limited in the culture; 2. Alginate is usually deemed as a cell adhesion resistant (CAR) material for cell attachment, therefore most anchorage-dependent cells are unable to attach and grow on the alginate surface. Therefore, its application is limited; 3. Mechanical strength of porous alginate carriers is low and susceptible to be degraded in an agitated culture environment, especially the culture medium containing sodium and potassium ion. Therefore, it would be limited to static culture environment and cannot be applied in large-scale production. As result of these limitations, alginic acid has never been a material used for anchorage-dependent cell culture in large production.

### SUMMARY OF THE INVENTION

In order to solve the aforementioned disadvantages of the microcarrier culture system in the prior art, which include high cost and high cell mortality rate due to exposing to high level of shearing forces caused by stirring and aeration during cultivation. The present invention provides a method to enhance the integrity of the growth surface by protecting the growth surface in a rigid, and porous solid layer.

In order to solve the aforementioned disadvantage of the porous microcarriers in the prior art in retaining cellular products that results in the adsorption of growth factors and other components from the medium. The present invention provides additional calcium ion in the culture media surrounding the alginate hydrogel surface to make anchorage-dependent cells adhere, spread and grow in a comparable growth rate and density.

In order to solve the aforementioned disadvantages of the carriers made by alginic acid (or alginate) in the prior art, which include: the cell density and viability can be limited in the culture; most anchorage-dependent cells are unable to attach and grow on the alginate surface; and the mechanical strength of porous alginate carriers is low. The present invention provides additional calcium ion in the culture media surrounding the alginate hydrogel surface to make anchorage-dependent cells adhere, spread and grow in a comparable growth rate and density, and protects the porous alginate matrix in netting, the porous growth matrix can remain its integrity for a long period of time during the cell culture.

In order to solve the disadvantages of the carriers for cell cultivation in the aforementioned prior art, which include: the smooth surface carriers (nonporous or poreless) does not lend itself to a large growth surface area and thus limits the number of cells to be adhered and grown; the task of harvesting cells from the porous carriers is often very difficult; and none of the carriers is capable of programming degradation and allowed releasing cells easily while retaining high surface-to-volume ratio and cell-adhesion properties for cell cultivation. The present invention provides a carrier which is porous and able to be degraded entirely and the cells are freed in the end of the culture. As a result, high cell density culture and high yield of cell harvest can be achieved and the process can be much simplified.

The present invention discloses a novel degradable growth surface and structure for culturing cells that maximizes cell attachment, enhances cell growth, increases mechanical strength, and increases cell density by significantly increasing the surface area by geometric manipulation. As a result, it can be applied in large-scale cell cultivation for cell/tissue mass production.

One object of the present invention is to provide a cultivating carrier system that can keep the mechanical strength of the porous carriers before programming degradation, it can be stacked on top of each other without overlapping to provide at least one three-dimensional space to facilitate the free and uniform flowing of the culture medium within the cultivation vessel or bioreactor. In addition, the unique degradable properties of the carrier system can facilitate cell or tissue harvest after the cell culture is completed.

The objects of the present invention include: providing a novel cell cultivating growth surface that are able to support cell adhesion and growth; providing a structure that is able to sustain mechanical stress during agitating culture environment and remain the integrity of the carrier; and providing a cell cultivating growth surface that is able to program degradation and facilitate tissue or cell mass harvest after the culture is completed.

To achieve the objects mentioned above, the present invention discloses a three-dimensional porous growth surface made from anionic polysaccharide material, especially alginic acid and/or its derivatives, to improve efficiency in culturing of anchorage-dependent cells, enhance cell growth surface, promote cell immobilization, promote cell propagation, maintain surface structure integrity, enable programmable degradation, and thus increase cellular production. The present invention teaches a method to enhance the integrity of the growth surface by protecting the growth surface in a rigid, and porous solid layer. The present invention further teaches a method of providing a favorable environment by employing a calcium ion concentration of >2.3 mM inside or surround the growth surface or in the culture medium. The modification includes the steps of increasing surface area by creating porous and 3-D structure, and treating the growth surface by increasing a calcium ion concentration inside or surround the growth surface or in the culture medium. The growth surface is uniquely capable of programming degradation and releasing the cell/tissue mass easily after the culture is completed.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The following description, given by way of example, is not intended to limit the present invention to any specific embodiment described. The description may be understood in conjunction with the accompanying Figures, incorporated herein by reference.

FIG. 1 shows a novel carrier structure of the present invention wherein the outer layer for porous carrier protection is three-dimensional V-shaped. Porous alginate gel is enclosed inside the V-shape supporting layer 100 and netting 102 on the surface.

FIG. 2 shows a novel carrier structure of the present invention wherein the outer layer for porous carrier protection is three-dimensional U-shaped. Porous alginate gel is enclosed inside the U-shape supporting layer.

FIG. 3 shows a novel carrier structure of the present invention wherein the outer layer for porous carrier protection is three-dimensional W-shaped. Porous alginate gel is enclosed inside the W-shape supporting layer.

FIG. 4 shows a novel carrier structure of the present invention wherein the outer layer for porous carrier protection is three-dimensional O-shaped, or a column. Porous alginate gel is enclosed inside the O-shape supporting layer.

FIG. 5 shows a novel carrier structure of the present invention wherein the outer layer for porous carrier protection is three-dimensional ( )-shaped. Porous alginate gel is enclosed inside the ( )-shape supporting layer.

FIG. 6 shows a novel growth surface of the present invention wherein the growth surface is bowl-shaped.

FIG. 7 shows the porous growth surface structure before cell culture.

FIG. 8 shows the cell growth morphology in the novel growth surface.

FIG. 9 shows the cell growth in a 2% alginate growth surface without further surface treatment and calcium ion reinforcement.

FIG. 10 shows the released cell/tissue after the degradation of the novel growth surface.

FIG. 11 is a schematic flowchart illustration the method of making a cell growth surface in accordance with one embodiment of the present invention.

### DESCRIPTION OF THE PREFERRED EMBODIMENT

In our laboratory, it is surprising to discover that by supplying additional calcium ions in the culture media surrounding the alginate hydrogel surface, many anchorage-dependent cells which normally could not adhere, spread and grow normally in alginate surface, could spread and grow on the surface in a comparable growth rate and density than that in conventional tissue culture plate. There is no requirement of further coating of any other extra-cellular matrix components such as collagen, fibronectin on the alginate surface to enhance the cell adhesion, spreading and growth process. In such design, the alginate growth surface could be programmable degraded by supplying ion chelater such as EDTA or sodium citrate. Another finding is that when making the alginate hydrogel a porous 3-D growth matrix, it is susceptible to be destroyed in an agitated culture environment. In order to solve the problem, we found that by protecting the porous alginate matrix in netting, the porous growth matrix can remain its integrity for a long period of time during the cell culture. These two major findings enable us to construct a growth matrix that are able to be applied in a dynamic culture environment and could support cell/tissue harvest without the requirement of enzymatic treatment. Nonetheless, the former finding was also found to be true to a non-porous or a porous alginate hydrogel matrix without protection by netting to which the integrity and rigidity of the surface may be sacrificed. It implied that the finding can be applied to any alginate hydrogel matrix with any means of protection or configuration.

Alginate, also known as alginic acid, are linear unbranched polymers containing beta (1-4)-linked D-mannuronic acid (M) and alpha-(1-4)-linked L-guluronic acid (G) residues. Alginates are able to be cold setting in the presence of calcium ions, or other multivalent metal ions such as Mg++, Sr++, and Ba++. Alginate gel can also easily be degraded by adding chelating agent such as sodium citrate, or EDTA. Therefore, it is an ideal material for release control such as drug release control. Alginate gel could also be able to form porous structure by common freeze/lyophilization procedure. Within the many advantages providing by the alginic acid material, it has been a common material utilizing in food, pharmaceutical and other industries. However, due to its cell adhesive resistance (CAR) nature, anchorage-dependent cells are very difficult to anchor and grow on the alginate surface. Therefore, even alginate has been applied in cell culture field for decades, but is only restricted in limited cell types and most of them are anchorage-independent cells, such as hybridoma. Nevertheless, if the alginate growth surface is further processed to be a porous structure, it would become very fragile in a solution containing sodium or potassium ions and is prone to be destroyed under agitating culture environment. As shown in Table 1, when placing the porous alginate structure in an agitating culture surface, only the one that are protected by 3-D folded netting according to present invention could remain its integrity for at least 10 days. The one without protection or with only one side protection will be disrupted within 24 hours. Therefore, it is very difficult to utilize porous alginate growth surface as a carrier for large-scale culture in a dynamic culture environment without further protection.

TABLE 1
Duration of the porous alginate structure placed
in an agitating culture surface.

| | Hours of Agitation Before |
|---|---|
| Growth Surface | Disrupted |
| Present Invention | No disruption after 10 days |
| Only one side protected | 24 hours |
| No protected | 2 hours |

The present invention, which teaches a three-dimensional porous growth surface made from polysaccharide material, especially alginic acid and/or its derivatives, is disclosed to enhance cell growth surface, promote cell immobilization, maintain surface structure integrity, enable programmable degradation, and thus increase cellular production. The present invention teaches a method to enhance the integrity of the growth surface by protecting the growth surface in a rigid, porous solid layer. The present invention further teaches a method of modifying a growth surface for eukaryotic and/or prokaryotic cells comprising the steps of increasing surface area by creating porous and 3-D structure, treating a surface to encourage cell attachment, promoting cell growth and proliferation and disposing the growth surface in any conventional cell cultivating device. The growth surface enables programmable degradation and releases the cell/tissue mass by adding chelating agent such as sodium citrate or EDTA after the culture is completed. The cell/tissue mass can also be further disassociated by adding trypsin, protease, collagenase and/or DNAse to obtain single cells.

The following detailed description, given by way of example, is not intended to limit the invention to any specific embodiment described. The detailed description may be understood in conjunction with the accompanying figures, incorporated herein by reference. Without wishing to unnecessarily limit the foregoing, the following shall disclose the present invention with respect to certain preferred embodiments. The embodiments in accordance with the present invention are suitable for prokaryotic and/or eukaryotic cell cultures and particularly for animal cells and/or mammalian cells. The present invention, inter alia, teaches a novel growth surface and structure suitable for culturing any cells that can sustain its mechanical strength for support cell growth, can be programmed to be degraded and be easy to harvest cell/tissue after the growth surface is degraded.

The novel growth surface according to the present invention is made from a combination of a rigid support plus a material that is biodegradable, flexible, yet sturdy and capable of maintaining any configuration given.

The novel growth surface according to the present invention is made from the following steps: first, construct a solid support to form a three-dimensional shape; second, submerge the solid support into an alginate solution and confine a certain amount of the alginate solution in the solid support; third, solidify the alginate solution through freezing, or cross-linking in multivalent metal ion solution such as calcium ion, magnesium ion, or barium ion, preferably calcium ion, to form a hydrogel; fourth, the pores in the alginate gel are formed by well-known freeze/lyophilization process before or after gel formation; fifth, supply with excess dication in the media inside or surrounding the growth surface and allow the growth surface to dry, or supply with excess dication, preferably calcium ion, and make total dication concentration greater than 2.3 mM in culture medium during culture.

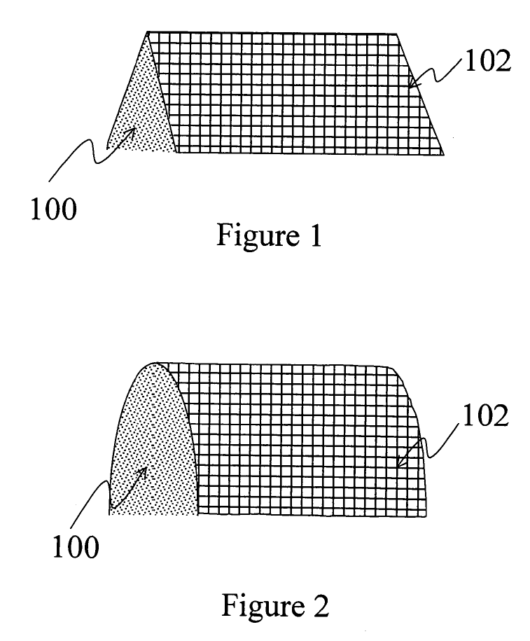

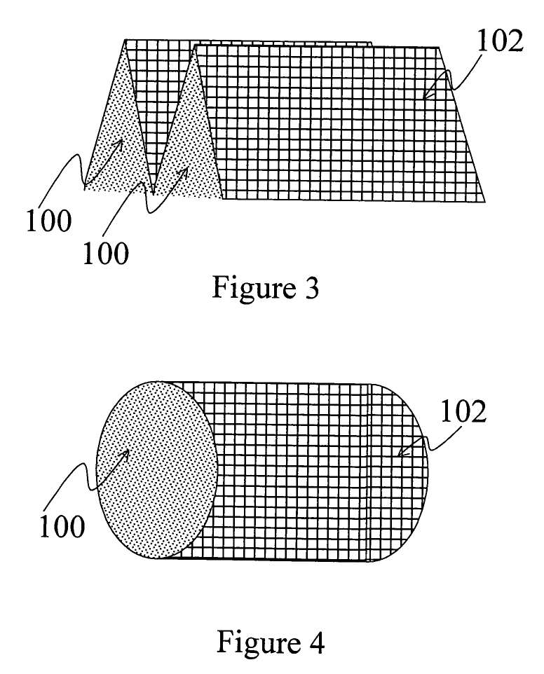

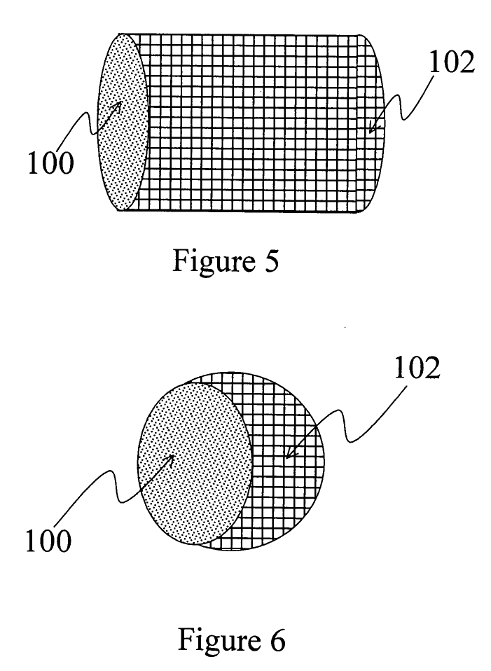

The rigid support, for example a netting or mesh made by polypropylene or nylon, is porous. The rigid support is bended or annealed to form a I, [ ], or V, or W, or U, or bowel, or ( ), O or any three-dimensional shape in order to be able to confine the porous growth surface inside the rigid support and protect the porous growth surface. Please refer to FIG. 1 to FIG. 6, they show a variety of novel carrier structures of the present invention, wherein the outer layers for porous carrier protection are three-dimensional V-shaped, U-shaped, W-shaped, O-shaped (or column-shaped), ( )-shaped and bowl-shaped, porous alginate gel is enclosed inside the supporting layer. In those figures, number 10, 20, 30, 40, 50 and 60 represent the “netting”, and number 15, 25, 35, 45, 55 and 65 represent the “porous growth surface”. The rigid support has at least two sides to cover the cell growth surface or has circling-portion to surround the cell growth surface to protect it from decomposition during cell culture and to provide mechanical strength that enables to stack each other.

The rigid support is porous, so that the cells/tissue could penetrate into the growth surface during inoculation, penetrate out of the growth surface after growth surface degradation, and could also facilitate the nutrient and oxygen to transfer into the growth surface. The rigid support may also be non-porous, so that it could be applied in a relatively static culture environment. The pore of the rigid support could be ranged from 500 um to 5 mm in diameter. More preferably, the pore of the rigid support could be ranged from 500 um to 2 mm in diameter.

The concentration of alginate solution can be ranged from 1% to 5%. The porous structure of the alginate hydrogel could also be constructed by other common practice for porous structure formation such as salt leaching, phase separation, or aphron freeze-and-dry. The preferable method is freeze/lyophilization and aphron freeze-and-dry. The pore size inside the porous structure could range from 10 um to 500 um. More preferably, the pore size could range from 50 um to 500 um. The pores in accordance with the present invention provide a maximum surface area to facilitate cell attachment, cell adhesion and cell proliferation, thereby provides a maximum cell density and thus, maximum cellular products.

Due to the inertness of the alginate surface to cell attachment and growth, so called a cell adhesion resistant (CAR) material, the growth surfaces are further reinforced by adding excess dication ions, such as calcium ion, before or during cell culture, and optionally coated with polycation polymers such as poly-L-lysine, poly-D-lysine, polyarginine, polyethyleneimine, poly-D-ornithine, or ploy-L-ornithine. More preferably, calcium ions are selected due to its economical and biocompatible feasibility. The alginate growth surface are further optionally coated with extra-cellular matrix, or attachment factors, such as collagens, fibronectin, laminins, trhombospondin 1, vitronectin, elastin, tenascin, or other cell adhesion molecules. However, the coating of attachment factors or extra-cellular matrix is not essential in present invention.

The novel growth surface of the present invention can be in any size, shape, form, structure or geometric configuration so long as it is in accordance with the spirit of the present invention. The growth surface of the present invention can be in any suitable form, such as a pellet, a strip, a ribbon, a spiral, a sheet, or any three-dimensional structure. In one embodiment, the growth surface of the present invention is in the form of a strip. The growth surface of the present invention may also be in the form of a pellet that can be of a variety of sizes having a diameter ranging from about 1 millimeter to about 250 millimeters, although any diameter may be deemed suitable depending on the individual needs. Preferably, the growth surface is in the form of pellets that are loosely packed as a matrix in a culture tank or a culture flask or a bioreactor. The porous carrier or growth surface or pellet can form a loosely packed bed that allows for easy and efficient distribution of the cells during inoculation and assures maximum cell adhesion on the surfaces of the porous pellet or porous growth surface or porous carrier.

One of skill in the art will understand that certain characteristics of a growth surface can have an effect on its performance. Carrier or surface characteristics, such as surface properties, carrier density, size, toxicity and rigidity can affect the performance of the growth surface and thus the performance of the cell culture particularly with respect to the cell density and the overall production of cellular products. Specifically, the size of the pores of the growth surfaces can affect the performance of the cells. Although one of ordinary skill in the art will appreciate that any growth surface pore size known will be suitable, the pore size is preferably in the range from 50 micrometer to 500 micrometers.

Nonetheless, the applied method in the surface is also important and critical for enhancing the overall performance, in particular, by retaining excess calcium ion concentration in the environment where the surface resides. This might be due to the potassium and sodium ion in the culture medium, which could replace the calcium ion and degrade the alginate surface and thus impede the cell spreading and propagation. Even most of the cell culture medium already contains around 1.8 mM (200 mg/L calcium chloride) calcium ions, however, it does not bring any benefits for promoting cell attachment and spreading on alginate growth surface. Only by further increasing the overall calcium ion concentration in the culture medium to above 2.3 mM, the cells start to show signs to attach and spread on the alginate growth surface. The cell attachment and spreading efficiency increased as the calcium ion concentration increased. The overall concentration of the calcium ion presented in the culture medium during culture is ranged from 2.3 mM to 300 mM, and more preferably ranged from 3 mM to 60 mM, and further preferably ranged from 3 mM to 10 mM. The present of excess dication ions, especially the calcium ions, on or inside alginate growth surface largely increase the types of anchorage-dependent cells that can be applied in the biodegradable material. The experimental data clearly supported the surprising results by using the unique growth surface and supporting condition disclosed in the present invention.

### EXAMPLES

### Example 1

### Preparing a Porous Growth Surface

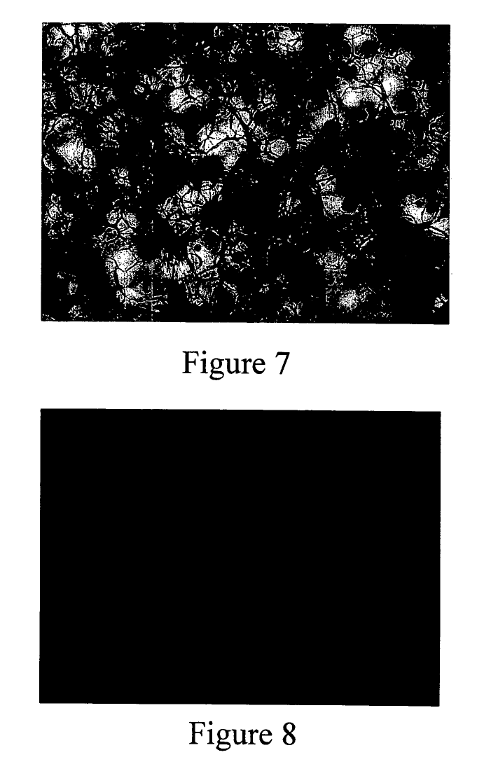

This example describes the manufacture of representative porous structures of the present invention. The porous structures described in this example are useful as scaffolds for physically supporting the growth of living cells. Material and Methods: PolyPropylene Netting with 1 m/m×1 m/m grid dimension was purchased from local store. Alginic acid powder was purchased from FMC BioPloymer (Philadelphia, Pa. 19103, USA). Calcium chloride was purchased from Sigma-Aldrich(www.sigmaaldrich.com). The netting was cut to 10 cm long×3 cm wide and was folded and heat-annealed to form a 3 dimensional ( ) shape column with width of 1 cm, and height of 3 mm. Alginic acid powder was dissolved in DI water to form 2% (w/v) solution. Place the ( ) shape netting in a container. Pour the alginate solution into the ( ) shape netting and allow the alginate solution to fill inside the netting support. Submerge the netting support containing 2% alginate solution in a 300 mM Calcium chloride and allow to gel for 30 minutes. The netting/gel was then brought to freezer at Celsius −80 degree for two hours, and dehydrated under vacuum. The pores were formed inside the gel and were an interconnected porous structure. The pore size is around 30˜200 um. Cut the netting/porous gel to 1 cm long pellets. The porous structure, as observed under light microscope, is shown in FIG. 7.

### Example 2

### Surface Modification

The netting/porous gel pellet was then rinsed with excess DI water containing 100 mM CaCl₂, and allowed the pellets to dry. The control group was prepared without adding excess CaCl₂ but just rinsed thoroughly with DI water to ensure no free calcium ion remain in the alginate surface and allowed drying. The growth structures were then sterilized under UV for over night.

### Example 3

### Cell Culture

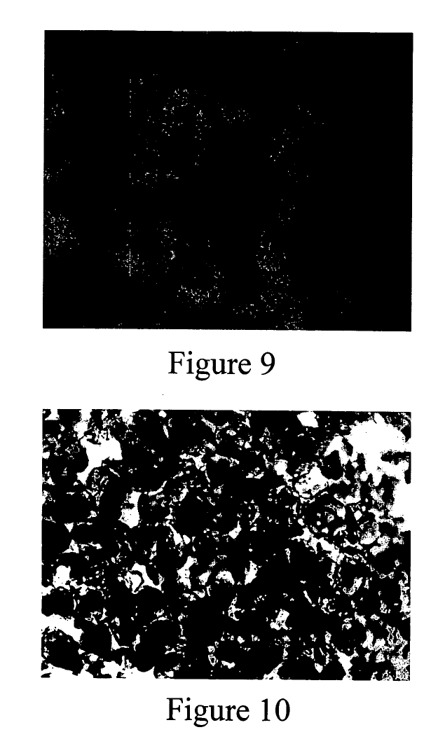

Prepare Vero cells (ATCC CCL-81) in M199/5% FBS. Place each porous alginate pellet in a well of a 12-well plate, seed with 1×10⁵ cells in each pellet and 2 ml culture medium. The calcium ion concentration in the one containing excess CaCl2 was diluted to around 10 mM with the culture medium before culture was initiated. On day 5th, fix the cells in one of the pellet by serial dehydration with 95% ethanol, and stain with Coomassie brilliant blue G. Observe the cell morphology under microscope. Cell morphology is shown in FIG. 8. It shows that Vero cells could propagate in the growth surface of present invention and fully occupy the growth space. In contrast to the control group (as shown in FIG. 9) without excess calcium ion appeared in the alginate pellet or in culture medium, cells are unable to adhere on the growth surface and will aggregate and fail to proliferate.

### Example 4

### Cell/Tissue Releasing

Take one pellet of present invention and submerge with 1.6% sodium citrate solution, shake for several minutes until the gel are dissolved, and cell tissue remained. Cells are found forming sheet or 3-D structure due to the 3-D porous structure of the pellet as shown in FIG. 10. Cell tissues are then centrifuge and re-suspend the pellet in enzymatic solution to dissociate the tissue to form single cells. 8.0×10⁵ cells are collected from one porous alginate carrier of the present invention, means around a 8 folds increase of cells within 5 days cultivation, which is within reasonable range for Vero cell propagation. In contrast to the control group (as shown in FIG. 9) without excess calcium ion appeared in the alginate pellet or in culture medium, cells are unable to adhere on the growth surface and will aggregate and the proliferation rate is slow and only 1.7 folds increased within same days of culture. It shows that the present invention could provide a cell growth surface and structure that are able to provide cell adhesion, cell propagation, and cell mass harvest in a biodegradable porous structure.

### Example 5

### Test with Other Anchorage-Dependent Cell Lines

The culture performance in the cell growth surface with present invention were further evaluated with different anchorage-dependent cell lines including Vero, MDCK, MDBK, BHK-21, CHO-k1, HEK-293, RK-13, and 3T3. The experiment results are shown in Table 2 below:

TABLE 2
Experiments results of culture performance for
different anchorage-dependent cell lines.

| | | Harvest | | | |
|---|---|---|---|---|---|
| | | from | | | |
| | | Growth | | | |
| | | surface | | Harvest from | |
| | Seed | with | | Control with | |
| Cell | (cells/ | present | Fold | only alginate | Fold |
| Line | matrix) | invention | increased | matrix | increased |
| Vero | 1 × 10⁵ | 8.0 × 10⁵ | 8.0 | 1.7 × 10⁵ | 1.7 |
| MDCK | 1 × 10⁵ | 9.6 × 10⁵ | 9.6 | 1.5 × 10⁵ | 1.5 |
| MDBK | 1 × 10⁵ | 10.4 × 10⁵ | 10.4 | 0.92 × 10⁵ | 0.92 |
| CHO-k1 | 1 × 10⁵ | 20.4 × 10⁵ | 20.4 | 13.9 × 10⁵ | 13.9 |
| BHK-21 | 1 × 10⁵ | 18.0 × 10⁵ | 18.0 | 2.8 × 10⁵ | 2.8 |
| RK-13 | 1 × 10⁵ | 7.6 × 10⁵ | 7.6 | 1.5 × 10⁵ | 1.5 |
| HEK293 | 1 × 10⁵ | 4.4 × 10⁵ | 4.4 | 1.8 × 10⁵ | 1.8 |
| 3T3 | 1 × 10⁵ | 1.28 × 10⁶ | 12.8 | 0.85 × 10⁵ | 0.85 |

Except the CHO that is not absolutely anchorage-dependent cell line, other cell lines show significant difference on growth between the two different matrices.

It indicates that the present invention does prove that the conventional concept of alginic acid as a cell adherence resistant (CAR) material is not appropriate. Instead, with proper treatment with the alginate growth surface with excess dication ions, it could cultivate almost all kinds of anchorage-dependent cell lines.

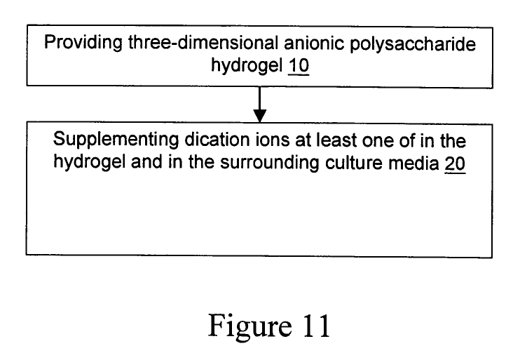

FIG. 11 is a schematic flowchart illustration the method of making a cell growth surface in accordance with one embodiment of the present invention. First, a three-dimensional hydrogel is prepared (step 10). In one embodiment, the three-dimensional hydrogle is prepared by cross-linking a polysaccharide polymer on a rigid support. Alternatively, a solid support is constructed to form a three-dimensional cavity first. Then the solid support is submerged into an alginated solution or mixture containing alginate and a certain amount of the alginate solution is confined in the solid support. Next, the alginate solution is solidified in dication solution to form a hydrogel, in which calcium ions may be used. Optionally, a plurality of pores may be formed inside the alginate hydrogel by freezing and lyophilizing the hydrogel. Alternatively, the cell surface of the hydrogel is modified by coating with non-covalent polycation. Alternatively, the pores may be formed by salt leaching the hydrogel. In another embodiment, the pores are formed by freezing and drying the hydrogel.

In another embodiment, the 3-D hydrogel is provided by preparing water-dispersible or water-soluble alginates (sodium alginate and calcium alginate), freeze drying the aqueous algin dispersion or gel to form a resulting algin sponge, and then lyophilizing the resulting algin sponge. In another embodiment, the 3-D hydrogel may be provided by dispersing a gas and alginate solution, freeze drying the aqueous solution or suspension to form foam-like structure of a resulting freeze-dried foam, and then lyophilizing the resulting freeze-dried foam. In another embodiment, the 3-D hydrogel may be made by mixing an aqueous solution of a water soluble alginate composition with a water soluble sequestering agent, adding a plasticizer and a surface active agent into the mixture, adding multi-valent metal ion to form water-insoluble alginate hydrogels, freezing the insoluble alginate hydrogel, and lyophilizing the frozen composite insoluble alginate hydrogel. Alternatively, the 3-D hydrogel may be prepared by providing a solution of a soluble polysaccharide in water, freezing the solution to form a frozen solution, cross-linking the frozen solution, and drying the resulting cross-linked and exchanging the polysaccharide material by solvent. Alternatively, the 3-D hydrogel may be derived from preparing a polysaccharide solution, subjecting the polysaccharide solution to gelation to get a polysaccharide gel, freezing the gel, and drying the frozen gel to obtain a polysaccharide sponge. In another embodiment, the 3-D hydrogel is employed by preparing a soluble alginate and gas emulsion, freezing and lyophilizing the soluble alginate and gas emulsion, cross-linking the frozen and lyophilized solution, and then again lyophilizing the solution. Accordingly, it is appreciated that the preparation of the 3-D hydrogel in the present invention is not limited to the formation aforementioned.

Second, excess dication ions are supplied with concentration greater than 2.3 mM during culture (step 20). In one embodiment, the dication ions are supplied in the hydrogel or in the surrounding culture media. Alternatively, the dication ions are supplied in the hydrogel and in the surrounding culture media where the cell growth surface resides.

The foregoing descriptions of specific embodiments of the present invention have been presented for purposes of illustrations and description. They are not intended to be exclusive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to particular use contemplated. It is intended that the scope of the invention be defined by the Claims appended hereto and their equivalents.

What is claimed is:

1. A method of making a cell growth surface to promote cell adherence, spreading and growth and to free cells or tissues by a programmable degradation, comprising:
providing a three-dimensional anionic polysaccharide hydrogel as the cell growth surface; and
supplementing excess dication ions with concentration greater than 2.3 mM at least one of in the three-dimensional anionic polysaccharide hydrogel and in a surrounding culture media where the cell growth surface resides.

2. The method of claim 1, wherein the providing step comprises cross-linking an anionic polysaccharide polymer to form the three-dimensional anionic polysaccharide hydrogel.

3. The method of claim 1, wherein the providing step comprises cross-linking an alginic acid or its derivatives to form the three-dimensional anionic polysaccharide hydrogel.

4. The method of claim 1, wherein the providing step comprises forming pores within the three-dimensional anionic polysaccharide hydrogel.

5. The method of claim 4, wherein the forming step comprises freezing and lyophilzing the three-dimensional anionic polysaccharide hydrogel.

6. The method of claim 1, wherein the dication ions are selected from the group consisting of calcium ions, magnesium ions, barium ions and the combination thereof.

7. The method of claim 1, wherein the dication ions are calcium ions.

8. The method of claim 7, wherein the calcium ion concentration is ranged from 2.3 mM to 300 mM.

9. The method of claim 7, wherein the calcium ion concentration is ranged from 3 mM to 60 mM.

10. The method of claim 7, wherein the calcium ion concentration is ranged from 3 mM to 10 mM.

11. A method of making a cell growth surface to promote cell adherence, spreading and growth and to free cells or tissues by a programmable degradation, comprising:
providing a rigid support;
solidifying an anionic polysaccharide polymer on the rigid support to form a three-dimensional hydrogel; and
supplementing excess dication ions with concentration greater than 2.3 mM in the three-dimensional hydrogel or in a surrounding culture media where the cell growth surface resides.

12. The method of claim 11, wherein the dication ions are supplemented in the hydrogel and in the surrounding culture media where the cell growth surface resides.

13. The method of claim 11, wherein the rigid support is porous or non-porous.

14. The method of claim 11, wherein the rigid support is porous and has a netting structure or a mesh structure.

15. The method of claim 14, wherein the netting structure and the mesh structure are made by polymer.

16. The method of claim 14, wherein the polymer to make the netting structure and the mesh structure is polypropylene or nylon.

17. The method of claim 11, wherein the rigid support has a [, U, V, W, ( ), O, bowl, or shovel shape to confine the cell growth surface inside the rigid support.

18. The method of claim 17, wherein the rigid support is porous and has at least two sides to cover the cell growth surface to protect it from decomposition during cell culture and to provide mechanical strength that enables to stack each other.

19. The method of claim 17, wherein the rigid support is porous and has circling-portion to surround the cell growth surface to protect it from decomposition during cell culture and provide mechanical strength that enables to stack on top of each other.

20. The method of claim 11, wherein the rigid support is non-porous and made by rigid biocompatible materials.

21. The method of claim 11, wherein the rigid support is non-porous and has a plate, or sheet shape.

22. The method of claim 11, wherein the polysaccharide polymer is alginic acid or its derivatives.

23. The method of claim 11, wherein the dication ions are calcium ions.

24. The method of claim 23, wherein the calcium ion concentration is ranged from 2.3 mM to 300 mM.

25. The method of claim 23, wherein the calcium ion concentration is ranged from 2.3 mM to 60 mM.

26. The method of claim 23, wherein the calcium ion concentration is ranged from 3 mM to 10 mM.

27. The method of claim 11, further comprising modifying the cell growth surface by coating with polycation.

28. The method of claim 27, wherein the polycation is selected from the group consisting of poly-L-lysine, poly-D-lysine, polyarginine, polyethyleneimine, poly-D-ornithine, ploy-L-ornithine and the combination thereof.

29. The method of claim 11, further comprising employing freezing and lyophilizing to form pores within the three-dimensional hydrogel.

30. The method of claim 11, further comprising employing salt leaching to form pores within the three-dimensional hydrogel,

31. The method of claim 11, further comprising freezing and drying the three-dimensional hydrogel to form pores within the three-dimensional hydrogel.

32. The method of claim 11, the rigid support has at least one fold or has at least one deformation to create at least one three-dimensional cavity.

33. The method of claim 11, wherein the method of making a cell growth surface is for eukaryotic cells.

34. The method of claim 11, wherein the method of making a cell growth surface is for anchorage-dependent cells.

35. The method of claim 11, further comprising culturing and harvesting cells.

36. The method of claim 35, wherein the culturing and harvesting step comprises:
randomly distributing the cell growth surface in a culture chamber;
culturing a plurality of cells on the cell growth surface;
disassociating the cell growth surface by adding a chelating agent after the cells culture is completed; and
harvesting the released cells by a separation means.

37. The method of claim 36, further comprising adding enzyme to disassociate the cells into isolated cells.

38. The method of claim 37, wherein the enzyme is selected from the group consisting of collagenase, trypsin and the combination thereof.

39. The method of claim 36, wherein the chelating agent is selected from the group consisting of sodium citrate, citric acid and the combination thereof.

40. The method of claim 36, wherein the separation means is a centrifuge.

41. The method of claim 11, wherein the solidifying step comprises freezing or cross-linking the anionic polysaccharide polymer with cation ions.
